(19) United States
(12) Patent Application Publication
(10) Pub. No.: US 2021/0341498 A1
(43) Pub. Date: Nov. 4, 2021

(54) BLOOD-CLOTTING-SYSTEM ANALYSIS APPARATUS, BLOOD-CLOTTING-SYSTEM ANALYSIS METHOD, AND BLOOD-CLOTTING-SYSTEM ANALYSIS PROGRAM

(71) Applicant: Sony Corporation, Tokyo (JP)

(72) Inventors: Yoshihito Hayashi, Chiba (JP); Seunmin Lee, Kanagawa (JP); Aya Fuchigami, Japan (JP); Kenzo Machida, Kanagawa (JP)

(73) Assignee: Sony Corporation, Tokyo (JP)

(21) Appl. No.: 17/271,540

(22) PCT Filed: Sep. 6, 2019
(86) PCT No.: PCT/JP2019/035270

§ 371 Date: Feb. 25, 2021

(30) Foreign Application Priority Data
Sep. 7, 2018 (JP) 2018-168346

(51) Int. Cl. G01N 33/86; G01N 27/02

(52) U.S. Cl. CPC G01N 33/86; G01N 27/026

(57) ABSTRACT

There is provided a blood-clotting-system analysis apparatus, including: a pair of electrodes; an application unit configured to apply alternating voltage to the pair of electrodes at predetermined time intervals; a measurement unit configured to measure a complex dielectric constant of a blood sample placed between the pair of electrodes; and an analysis unit configured to analyze, by using at least two or more types of blood clotting-related assays, activity of a clotting factor and activity of a clotting inhibition factor on a basis of a complex dielectric constant of a particular frequency in a predetermined time period measured at the time intervals after an anticoagulant effect acting on the blood sample is resolved.

### CROSS REFERENCE TO RELATED APPLICATIONS

This application claims the benefit of Japanese Priority Patent Application JP 2018-168346 filed Sep. 7, 2018, the entire contents of which are incorporated herein by reference.

### TECHNICAL FIELD

The present technology relates to a blood-clotting-system analysis apparatus, a blood-clotting-system analysis method, and a blood-clotting-system analysis program.

### BACKGROUND ART

In the past, there has been a blood clotting test as a method of analyzing the blood condition performed clinically. As a general blood clotting test, blood clotting tests typified by prothrombin time-international normalized ratio (PT-INR) and activated partial thromboplastin time (APTT) have been known. In these methods, clotting re-activity by proteins relating to a clotting reaction is analyzed. Such proteins are contained in plasma obtained by centrifuging the blood sample.

The above-mentioned test method is used for evaluating the functional test of extrinsic clotting ability and intrinsic clotting ability. In these test, substances that trigger an extrinsic clotting reaction and an intrinsic clotting reaction are added in excess to obtain the test result in a short time. These tests are performed using plasma obtained by centrifuging a blood sample. In such test, there occurs a discrepancy between the test result and the actual clinical condition in many cases because cellular components such as platelets and red blood cells, which play an important role in the blood clotting reaction in vivo are removed by centrifugation.

Note that examples of another functional test include thromboelastography and thromboelastometry, which are commercialized respectively as TEG (registered trademark) and ROTEM (registered trademark). These functional tests are not widespread enough because, for example, (1) measurement is not automated and the test result depends on the measurer's technique, (2) it is susceptible to vibration, (3) a quality control (QC) procedure is complicated and a QC reagent is expensive, and (4) it is necessary to acquire a skill to interpret an output signal (thromboelastogram). Further, there is a possibility that the needs of the medical field are not satisfied because sensitivity to deficiency or inhibition of the extrinsic and intrinsic clotting factors is not shown so much.

Further, in the above-mentioned existing functional test, it is usual to use a clotting initiator as a test reagent in an excess amount larger than that of the reaction in vivo. Therefore, these tests are suitable for evaluating the remarkable reduction in clotting ability, i.e., the bleeding tendency, but are not suitable for evaluating the remarkable increase in clotting ability, i.e., the thrombotic tendency, or a slight change in blood clotting ability. Note that in the thromboelastometry, there is an assay that performs measurement by re-adding calcium without using a clotting initiator that artificially activates the extrinsic clotting pathway or the intrinsic clotting pathway. In the assay, fibrin gel formed early in the clotting reaction is broken due to rotational displacement during measurement, and thus, it is difficult to perform correct measurement. In particular, in such an assay, there has been a problem that repeatability of measurement is not expected for samples with low levels of fibrinogen or platelets.

In recent years, as another method capable of evaluating blood clotting measurement simply and accurately, a method of performing dielectric measurement of the blood clotting process is devised (see, for example, Japanese Patent Application Laid-open No. 2010-181400 and Japanese Patent Application Laid-open No. 2012-194087). In this method, a blood sample is filed in a capacitor-like sample section including a pair of electrodes and the like, and an alternating electric field is applied to the sample section to measure the change in the complex dielectric constant, which occurs in the clotting process of the blood sample. Y. Hayashi et al., Analytical Chemistry 87(19), 10072-10079 (2015) shows that by using this method, the process of clotting and fibrinolysis can be easily monitored. Further, I. Uchimura et al., Biorheology 53, 209-219 (2016) shows that by using the method, it is possible to evaluate the increase in blood clotting ability with high sensitivity, which has been difficult to evaluate by another method. However, also the method has a problem that it is extremely difficult to correctly evaluate complicated blood clotting kinetics.

### CITATION LIST

### Patent Literature

PTL 1: Japanese Patent Application Laid-open No. 2010-181400
PTL 2: Japanese Patent Application Laid-open No. 2012-194087

### Non Patent Literature

NPL 1: Y. Hayashi et al., Analytical Chemistry 87(19), 10072-10079 (2015)
NPL 2: I. Uchimura et al., Biorheology 53, 209-219 (2016)

### SUMMARY

### Technical Problem

As described above, with the existing method, it has been extremely difficult to correctly evaluated complicated blood clotting kinetics.

In this regard, it is desired to provide a blood-clotting-system analysis apparatus capable of evaluating blood clotting kinetics with high precision.

### Solution to Problem

According to the present disclosure, there is provided a blood coagulation system analysis device. The blood coagulation system analysis devices comprises circuitry configured to perform a first blood clotting-related assay on a first blood sample, perform a second blood clotting-related assay on a second blood sample, and evaluate an activity of a clotting factor and an activity of a clotting inhibition factor based, at least in part, on an output of the first blood clotting-related assay and an output of the second blood clotting-related assay, wherein the first blood sample and the second blood sample are distributed from a same blood sample collected from a patient.

According to the present disclosure, there is provided a method of analyzing a blood sample to determine an amount of a thrombosis risk and/or an amount of a bleeding risk of a patient associated with the blood sample. The method comprises performing a first blood clotting-related assay on a first blood sample, performing a second blood clotting-related assay on a second blood sample, and evaluating an activity of a clotting factor and an activity of a clotting inhibition factor based, at least in part, on an output of the first blood clotting-related assay and an output of the second blood clotting-related assay, wherein the first blood sample and the second blood sample are distributed from a same blood sample collected from a patient.

According to the present disclosure, there is provided a blood coagulation analysis system. The blood coagulation analysis system comprises a first electrode and a second electrode arranged opposite the first electrode such that a container including a first blood sample may be arranged between the first and second electrodes, a voltage generator configured to apply an alternating voltage to the first electrode and the second electrode, and circuitry. The circuitry is configured to perform a first blood clotting-related assay on the first blood sample when the alternating voltage is applied to the first and second electrodes, perform a second blood clotting-related assay on a second blood sample, and evaluate an activity of a clotting factor and an activity of a clotting inhibition factor based, at least in part, on an output of the first blood clotting-related assay and an output of the second blood clotting-related assay, wherein the first blood sample and the second blood sample are distributed from a same blood sample collected from a patient.

### BRIEF DESCRIPTION OF DRAWINGS

FIG. 1 is a schematic conceptual view schematically showing an example of the concept of a blood-clotting-system analysis apparatus 100 according to an embodiment of the present technology.

FIG. 2 is a cross-sectional view schematically showing an example of an electrical measurement container 101 according to an embodiment of the present technology.

FIG. 3 is a drawing-substitute graph describing an example of measurement of the complex dielectric constant spectrum (three-dimensional).

FIG. 4 is a drawing-substitute graph describing an example of measurement of the complex dielectric constant spectrum (two-dimensional).

FIG. 5 is a drawing-substitute graph describing an example of the feature amount extracted from the complex dielectric constant spectrum.

FIG. 6A to FIG. 6D are each a drawing-substitute graph showing changes in a screening clotting test and a clotting fibrinolytic factor with blood collection timing.

FIG. 7E to FIG. 7H are each a drawing-substitute graph showing changes in a screening clotting test and a clotting fibrinolytic factor with blood collection timing.

FIG. 8A and FIG. 8B are each a drawing-substitute graph showing changes in a ROTEM measurement result with blood collection timing.

FIG. 9C to FIG. 9E are each a drawing-substitute graph showing changes in a ROTEM measurement result with blood collection timing.

FIG. 10A to FIG. 10D are each a drawing-substitute graph showing changes in a DBCM measurement analysis result with blood collection timing.

FIG. 11E to FIG. 11H are each a drawing-substitute graph showing changes in a DBCM measurement analysis result with blood collection timing.

FIG. 12 is a block diagram showing an example of a blood clotting analysis method in consideration of thoroughly finding a thrombosis risk.

FIG. 13 is a block diagram showing an example of a blood clotting analysis method for determining a bleeding risk using an extrinsic weak triggering assay and an intrinsic weak triggering assay.

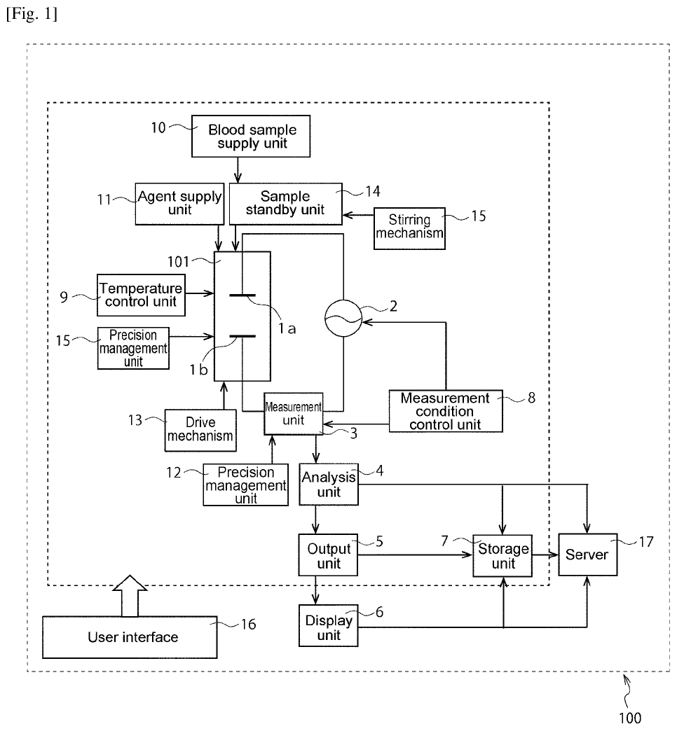

### DESCRIPTION OF EMBODIMENTS

Hereinafter, a favorable embodiment of the present technology will be described with reference to the drawings.

The embodiment described below shows an example of a typical embodiment of the present technology, and the scope of the present technology is not narrowly interpreted by the embodiment. Note that description will be made in the following order.

1. blood-clotting-system analysis Apparatus 100

(1) Pair of Electrodes 1a and 1b

(1-1) Electrical Measurement Container 101

(1-2) Connection Unit 102

(1-3) Container Holding Unit 103

(2) Application Unit 2

(3) Measurement Unit 3

(4) Analysis Unit 4

(5) Output Unit 5

(6) Display Unit 6

(7) Storage Unit 7

(8) Measurement Condition Control Unit 8

(9) Temperature Control Unit 9

(10) Blood Sample Supply Unit 10

(11) Agent Supply Unit 11

(12) Precision Management Unit 12

(13) Drive Mechanism 13

(14) Sample Standby Unit 14

(15) Stirring Mechanism 15

(16) User Interface 16

(17) Server 17

(18) Others

2. Blood Clotting Analysis Method

(1) Application Step

(2) Measurement Step

(3) Analysis Step

1. Blood-Clotting-System Analysis Apparatus 100

A blood-clotting-system analysis apparatus 100 includes at least a pair of electrodes 1a and 1b, an application unit 2, a measurement unit 3, and an analysis unit 4. Further, the blood-clotting-system analysis apparatus 100 may include, as necessary, other units such as an output unit 5, a display unit 6, a storage unit 7, a measurement condition control unit 8, a temperature control unit 9, a blood sample supply unit 10, an agent supply unit 11, a precision management unit 12, a drive mechanism 13, a sample standby unit 14, a stirring mechanism 15, a user interface 16, and a server 17. Hereinafter, details thereof will be described.

(1) Pair of Electrodes 1a and 1b

The pair of electrodes 1a and 1b are brought into contact with a blood sample B at the time of measurement, and apply necessary voltage to the blood sample B.

The arrangement, form, and the like of the pair of electrodes 1a and 1b are not particularly limited, and can be freely designed as necessary as long as it is capable of applying necessary voltage to the blood sample B. In the present technology, it is favorable that the pair of electrodes 1a and 1b are integrally formed in an electrical measurement container 101 described below.

Also the material forming the electrodes 1a and 1b is not particularly limited. One or two or more types of known electrically conductive materials can be freely selected and used as necessary as long as the condition and the like of the blood sample B to be analyzed are not affected. Specifically, examples of such material include titanium, aluminum, stainless steel, platinum, gold, copper, and graphite.

In the present technology, it is favorable to form the electrodes 1a and 1b of an electrically conductive material containing titanium, among these materials. Titanium is suitable for performing measurement of the blood sample B because it has a property of having low clotting activity with respect to a blood sample.

(1-1) Electrical Measurement Container 101

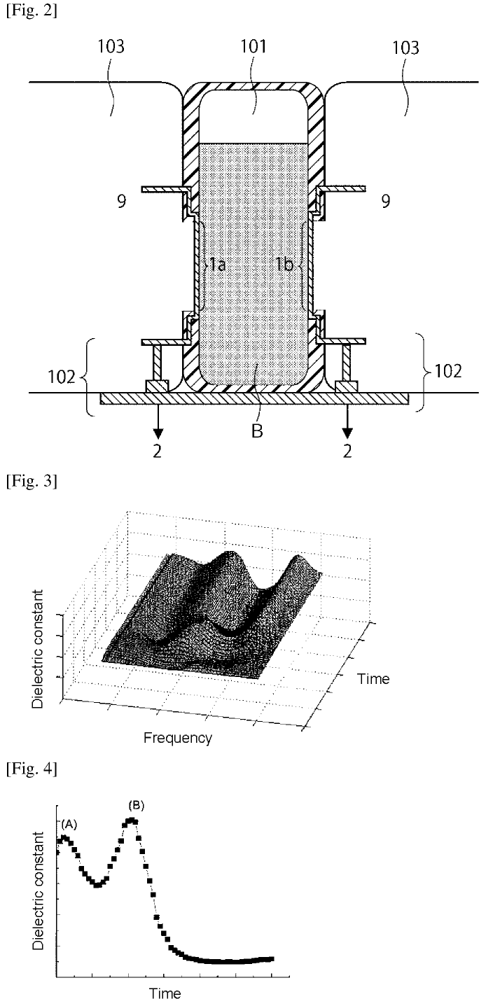

FIG. 2 is a cross-sectional view schematically showing an example of the electrical measurement container 101 according to an embodiment of the present technology. The electrical measurement container 101 holds the blood sample B to be analyzed. In the blood-clotting-system analysis apparatus 100 according to the embodiment of the present technology, the number of electrical measurement containers 101 is not particularly limited, and one or a plurality of electrical measurement containers 101 can be freely arranged as appropriate depending on the amount, type, and the like of the blood sample B to be analyzed.

In the blood-clotting-system analysis apparatus 100 according to the embodiment of the present technology, measurement of the complex dielectric constant is performed while the electrical measurement container 101 holds the blood sample B. For this reason, it is favorable that the electrical measurement container 101 is configured to be sealable when holding the blood sample B. However, the electrical measurement container 101 does not necessarily need to be configured to be airtight as long as it can be stationary for the time taken to measure the complex dielectric constant and the configuration does not affect the measurement.

The specific method of introducing the blood sample B into the electrical measurement container 101 and the sealing method are not particularly limited. The blood sample B can be introduced by a free method as appropriate depending on the form and the like of the electrical measurement container 101. Examples of such a method include a method of providing a lid in the electrical measurement container 101 and closing the lid for sealing after introducing the blood sample B by using a pipet or the like.

The form of the electrical measurement container 101 is not particularly limited and can be freely designed as appropriate as long as the blood sample B to be analyzed can be held in the apparatus. Further, the electrical measurement container 101 may include one or a plurality of containers.

The specific form of the electrical measurement container 101 is not particularly limited and can be freely designed as appropriate depending on the condition and the like of the blood sample B as long as the electrical measurement container 101 is capable of holding the blood sample B to be analyzed. Examples of such a form include a cylindrical body, a polygonal cylindrical body whose cross section is a polygon (triangle, square, or more), a cone, a polygonal pyramid whose cross section is a polygon (triangle, square, or more), and the form obtained by combining one or two or more of them.

Further, also the material forming the container 101 is not particularly limited and can be freely selected as appropriate as long as the condition and the like of the blood sample B to be analyzed are not affected. In particular, in the present technology, it is favorable that the container 101 is formed of a resin from the viewpoint of ease of processing and forming, and the like. In the present technology, the type and the like of the resin that can be used are not particularly limited. One or two or more types of resins that can be used for holding the blood sample B can be freely selected as appropriate and used. Examples of such a resin include hydrophobic and insulating polymers such as polypropylene, polymethyl methacrylate, polystyrene, acrylic, polysulfone, and polytetrafluoroethylene, copolymers, and blend polymers.

In particular, in the present technology, it is favorable that the electrical measurement container 101 is formed of one or more types of resins selected from the group consisting of polypropylene, polystyrene, acrylic, and polysulfone, among these. These resins are suitable for performing measurement on a blood sample because they have a property of having low clotting activity with respect to the blood sample.

Note that in the present technology, a well-known disposable cartridge type contained can also be used as the electrical measurement container 101.

In the present technology, it is favorable that the plurality of electrical measurement containers 101 including two or more types of blood clotting-related assays described below are provided. As a result, it is possible to efficiently perform analysis of the magnitude of the thrombosis risk in the analysis unit 4 described below. Further, in the present technology, it is favorable that reagents constituting the two or more types of blood clotting-related assays are encapsulated for each assay in each of the plurality of electrical measurement containers 101 in advance.

In the present technology, in the case of using an agent, as described above, a predetermined agent may be housed in the electrical measurement container 101 in advance in a solidified state, or in a liquid state as it is. For example, an anticoagulant, a clotting initiator, an intrinsic clotting pathway initiator, an extrinsic clotting pathway initiator, a calcium salt, and the like may be put in the container 101 in advance. By causing the container 101 to house an agent in advance as described above, the agent supply unit 11 described below or a section holding an agent are unnecessary, which makes it possible to miniaturize the apparatus and reduce the cost. Further, the usability can be improved because the user does not need to change the agent and maintenance of a device such as the agent supply unit 11 and a section holding an agent is unnecessary.

(1-2) Connection Unit 102

A connection unit 102 electrically connects the application unit 2 described below and the electrodes 1a and 1b. The specific form of the connection unit 102 is not particularly limited and can be freely designed as appropriate as long as it is capable of electrically connecting the application unit 2 and the electrodes 1a and 1b.

(1-3) Container Holding Unit 103

A container holding unit 103 holds the electrical measurement container 101. The specific form of the container holding unit 103 is not particularly limited and can be freely designed as appropriate as long as it is capable of holding the container 101 housing the blood sample B to be analyzed.

Further, also the material forming the container holding unit 103 is not particularly limited and can be freely selected as appropriate depending on the form and the like of the electrical measurement container 101.

Further, in the present technology, the container holding unit 103 may have a function (e.g., barcode reader, etc.) of automatically reading information relating to the container 101 from an information recording medium provided in the electrical measurement container 101. Examples of the information recording medium include an IC card, an IC tag, a card with a barcode or matrix 2D code, or a paper or sticker on which a barcode or matrix 2D code is printed.

(2) Application Unit 2

The application unit 2 applies alternating voltage to the pair of electrodes 1a and 1b at predetermined intervals. More specifically, for example, the application unit 2 applies alternating voltage to the pair of electrodes 1a and 1b at the start time of the time point when an instruction to start measurement is received or the time point when the power of the apparatus 100 is turned on. More specifically, the application unit 2 applies alternating voltage of a set frequency or a frequency controlled by the measurement condition control unit 8 to be described later to the pair of electrodes 1a and 1b for each set measurement interval or a measurement interval controlled by the measurement condition control unit 8 to be described later.

(3) Measurement Unit 3

The measurement unit 3 measures the complex dielectric constant of a blood sample placed between the pair of electrodes 1a and 1b. The configuration of the measurement unit 3 can be freely designed as appropriate as long as the measurement unit 3 is capable of measuring the complex dielectric constant to be measured of the blood sample B. Specifically, as the measurement unit 3, an impedance analyzer, a network analyzer, or the like can be adopted.

More specifically, for example, the measurement unit 3 may be configured to chronologically measure the impedance of the blood sample B obtained by application alternating voltage to the blood sample B by the application unit 2, and chronologically measure the impedance of the blood sample B between the electrodes 1a and 1b at the start time of the time point when an instruction to start measurement is received or the time point when the power of the apparatus 100 is turned on. Then, the complex dielectric constant is derived from the measured impedance. For the derivation of the complex dielectric constant, a known function or relation showing the relationship between the impedance and the dielectric constant can be used.

The measurement result by the measurement unit 3 can be obtained as a three-dimensional complex dielectric constant spectrum with the frequency, time, and the dielectric constant as coordinate axes (FIG. 3), or a two-dimensional complex dielectric spectrum with two of the frequency, time, and the dielectric constant as coordinate axes (FIG. 4). The Z axis in FIG. 3 indicates the real part of the complex dielectric constant in each time and each frequency.

FIG. 4 corresponds to a two-dimensional spectrum obtained by cutting the three-dimensional spectrum shown in FIG. 3 by the frequency of 760 kHz. The symbol (A) in FIG. 4 indicates a peak associated with rouleaux formation of red blood cells, and the symbol (B) indicates a peak associated with the blood sample clotting process. The inventors of the present invention disclose in Japanese Patent Application Laid-open No. 2010-181400 that the temporal change of the dielectric constant of the blood sample reflects the clotting process of the blood sample. Therefore, the complex dielectric constant spectrum obtained by the measurement unit 3 is an index quantitatively indicating the clotting ability of the blood sample. On the basis of the change in such an index, information relating to the clotting ability of the blood sample, such as the blood clotting time, blood clotting rate, and blood clotting strength, can be acquired.

(4) Analysis Unit 4

The analysis unit 4 uses at least two or more types of blood clotting-related assays, and analyzes activity of the clotting factor and activity of the clotting inhibition factor on the basis of the complex dielectric constant of a specific frequency in a predetermined time period, which is measured at the above-mentioned intervals from the time when the anticoagulant effect acting on the blood sample B has been resolved. Note that in the present specification, the concept of “activity” includes physiological activity including reduction in reaction rate.

As described above, it has become apparent in the process of examining the present technology that it is extremely difficult to correctly evaluate complicated blood clotting kinetics with the existing method. Specifically, as shown in Example 1 described below, it has been found that the thrombosis risk cannot be correctly analyzed with the existing method in the case where (i) the clotting ability is partially reduced due to deficiency of clotting factors but hypercoagulability is observed in relation to other factors of the like, or (ii) the cause of hypercoagulability is due to reduction in practical activity of the clotting inhibition factor (particularly, antithrombin) under physiological conditions.

The thrombosis risk in the case of (i) above is one that is found during recovery after surgery using cardiopulmonary bypass and cannot be found by a normal test. Further, in the case of (ii) above, if a vitamin K antagonist inhibitor (e.g., warfarin) generally prescribed to prevent thrombosis from occurring is used, the amount of antithrombin or the like, which is the clotting inhibition factor, is also reduced. Also in this case, a clotting test such as a PT-INR test cannot reflect reduction in the amount of antithrombin due to the characteristics of the test, which leads to the test result of clotting extension, and it is erroneously determined that the thrombosis risk has been reduced. However, in fact, both of the clotting ability and the ability to suppress clotting (mainly by antithrombin) under physiological conditions are reduced, and the balance between them is important for the thrombosis risk. Therefore, it cannot be necessarily said that the thrombosis risk is reduced. In order to prevent such a misunderstanding from occurring, it is necessary to perform not only the clotting test but also antithrombin activity measurement.

However, even if the antithrombin activity measurement is performed, the following case cannot be dealt with. Specifically, the case where the amount (concentration) of antithrombin is not reduced but a condition in which the physiological (dynamic) activity is reduced is a problem. In other words, the case where the amount of antithrombin is not changed but the reaction rate of antithrombin is reduced cannot be dealt with. The existing antithrombin activity test or quantitative test cannot reflect a slight change in the reaction rate of antithrombin. Meanwhile, in the case where whether or not thrombus is generated in vivo is a problem, not only the amount of antithrombin but also the reaction rate of antithrombin is important because the clotting factor activity reaction in which thrombin generation is accelerated and the reaction in which thrombin generation is inhibited compete. Such a thrombosis risk relating to the reaction speed of antithrombin has not been generally recognized until now.

Meanwhile, in particular, the blood-clotting-system analysis apparatus according to the embodiment of the present technology is capable of finding even a sample having the above-mentioned thrombosis risk without exception. Specifically, it is possible to evaluate also the thrombosis risk that have not been recognized until now, and evaluate blood clotting kinetics with high precision. As a result, it is possible to make tailor-made treatment that matches the clotting state of a patient in all diseases where thrombosis hemostasis control or clotting abnormality after surgery is a problem.

Further, in the condition in which reduction in clotting factor and reduction in activity of the clotting inhibition factor coexist, although not only a clotting test but also measurement of a blood clotting factor or the like need to be performed to estimate both of the risks in the case of the existing method, both of the risks can be easily evaluated by the present technology and treatment poly based on the risk determination result can be decided. Further, by applying the present technology to general medical checkup, it is possible to know the risk before thrombosis, cerebral hemorrhage, or the like occurs.

In the present technology, it is favorable the above-mentioned two or more types of blood clotting-related assays are any two or more assays selected from the group consisting of an assay that triggers no blood clotting reaction, an intrinsic-clotting-pathway triggering assay (hereinafter, referred to also as “intrinsic strong triggering assay”), an assay (hereinafter, referred to also as “intrinsic weak triggering assay”) that triggers an intrinsic clotting pathway more weakly than the intrinsic-clotting-pathway triggering assay, an extrinsic-clotting-pathway triggering assay (hereinafter, referred to also as “extrinsic strong triggering assay”), and an assay (hereinafter, referred to also as “extrinsic weak triggering assay”) that triggers an extrinsic clotting pathway more weakly than the extrinsic-clotting-pathway triggering assay.

Further, in the present technology, it is favorable that the two or more types of blood clotting-related assays include the intrinsic weak triggering assay and/or the extrinsic weak triggering assay.

The intrinsic strong triggering assay includes, for example, a calcium salt to resolve the anticoagulant effect by citric acid, and an intrinsic clotting pathway initiator at a concentration of strongly triggering the intrinsic clotting pathway. Examples of the intrinsic clotting pathway initiator include ellagic acid. In this case, for example, among general APTT test reagents, those using ellagic acid as a clotting activator (e.g., actin SFL) can be used. Regarding the final concentration of ellagic acid, the ratio of the blood sample:APTT test reagent is favorably 18:1 to 200:1, and more favorably 50:1 to 150:1, and the optimal final concentration is 90:1.

The intrinsic weak triggering assay includes, for example, a calcium salt to resolve the anticoagulant effect by citric acid, and an intrinsic clotting pathway initiator at a concentration of weakly triggering the intrinsic clotting pathway. Examples of the intrinsic clotting pathway initiator include ellagic acid. In this case, regarding the final concentration of ellagic acid, the ratio of the blood sample:APTT test reagent is favorably 230:1 to 2300:1, and more favorably 450:1 to 1800:1, and the optimal final concentration is 900:1.

The above-mentioned extrinsic strong triggering assay includes, for example, a calcium salt for resolving the anticoagulant effect by citric acid and an extrinsic clotting pathway initiator at a concentration of strongly triggering the extrinsic clotting pathway. Examples of the extrinsic clotting pathway initiator include a tissue factor (TF). In this case, the final concentration of the tissue factor is favorably higher than 5 pM, and more favorably not less than 10 pM, and the optimal final concentration is 50 pM.

The above-mentioned extrinsic weak triggering assay includes, for example, a calcium salt for resolving the anticoagulant effect by citric acid and an extrinsic clotting pathway initiator at a concentration of weakly triggering the extrinsic clotting pathway. Examples of the extrinsic clotting pathway initiator include a tissue factor. In this case, the final concentration of the tissue factor is favorably not more than 5 pM, and more favorably 0.2 to 2.0 pM, and the optimal final concentration is 0.6 to 0.7 pM.

In the present technology, the clotting factor whose activity is to be analyzed by the analysis unit 4 is not particularly limited, but can be particularly an intrinsic clotting factor as shown in Example 2 described below. Further, the clotting inhibition factor whose activity is to be analyzed by the analysis unit 4 is also not particularly limited, but can be particularly antithrombin as shown in Example 2 described below.

More specifically, the analysis unit 4 extracts the feature amount of the complex dielectric constant spectrum of a particular frequency measured using a certain assay, and determines, on the basis of, for example, the measurement result of a certain number of healthy persons and/or patients, whether or not the feature amount exceeds the determination reference set in advance. As the determination reference, for example, a threshold value may be simply set. However, it is favorable to use a value defined on the basis of a function of a blood clotting time (s) and a blood clotting time (w). The blood clotting time (s) is measured using a strong triggering assay, and the blood clotting time (w) is measured using a weak triggering assay.

Similarly, the analysis unit 4 extracts the feature amount of the complex dielectric constant spectrum of a particular frequency measured using another assay different from the above-mentioned certain assay, and determines whether or not the feature amount exceeds the determination reference.

Then, the analysis unit 4 compares the determination result obtained by using the certain assay with the determination result obtained by using the different assay, and performs classification on the basis of the determination results to finally determine whether the condition of each sample (each blood sample), i.e., the activity of the clotting factor and the activity of the clotting inhibition factor, is normal or reduced.

Note that as the above-mentioned feature amount, a temporal index relating to a blood sample clotting reaction, an index relating to the rate of the reaction, or the like can be adopted. Further, in the present technology, a new feature amount or value may be calculated by combining the feature amount by the certain assay and the feature amount by the different assay, and may be compared with the determination reference set in advance.

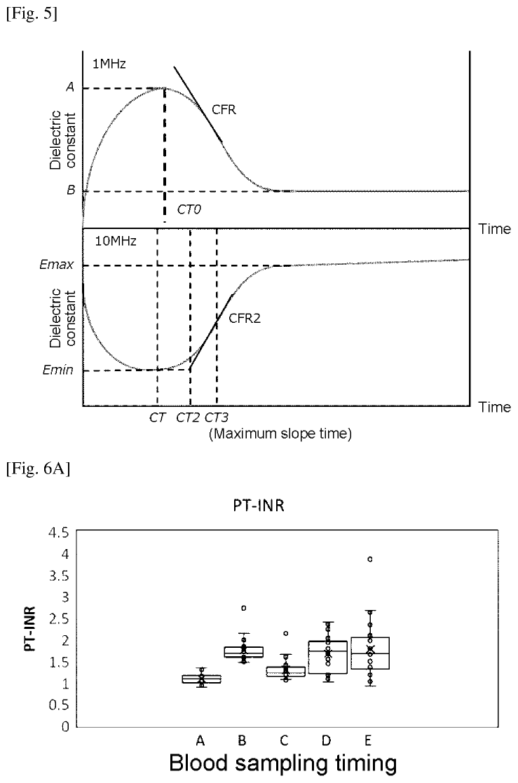

FIG. 5 is a drawing-substitute graph describing an example of the feature amount extracted from the complex dielectric constant spectrum. In FIG. 5, the vertical axis indicates the dielectric constant, and the horizontal axis indicates time. The upper graph is based on the measurement result near a frequency of 1 MHz (not less than 100 kHz to less than 3 MHz). The lower graph is based on the measurement result near a frequency of 10 MHz (3 to 30 MHz).

In the present technology, as the above-mentioned feature amount, the time feature amount and/or the slope feature amount extracted from the complex dielectric constant spectrum at the particular frequency can be used. Further, the slope feature amount may be extracted on the basis of the time feature amount extracted from the complex dielectric constant spectrum at the particular frequency. More specifically, as the feature amount, for example, one or more selected from the group consisting of a time CT0 that gives the maximum value of the complex dielectric constant at a low frequency not less than 100 kHz and less than 3 MHz, a time CT1 (not shown) that gives the maximum slope at the low frequency, a maximum slope CFR at the low frequency, a time CT4 (not shown) when the absolute value of the slope reaches a predetermined ratio (favorably, 50%) of the maximum slope CFR after the time CT1, a time CT that gives the minimum value of the complex dielectric constant at a high frequency of 3 to 30 MHz, a time CT3 that gives the maximum slope at the high frequency, a maximum slope CFR2 at the high frequency, a time CT2 that gives the minimum value of the complex dielectric constant when a straight line is drawn from the time CT3 with the slope of the maximum slope CFR2 after the time CT and before the time CT3, and a time CT5 (not shown) when the absolute value of the slope reaches a predetermined ratio (favorably, 50%) of the maximum slope CFR2 after the time CT3 can be used. Further, an operation value of these feature amounts or an operation value with the measured complex dielectric constant or the like can also be used. Additionally, analysis unit 4 can also analyze activity of the clotting factor and activity of the clotting inhibition factor by using a trained model comprising the model trained using one or more machine learning techniques.

(5) Output Unit 5

The output unit 5 outputs the analysis result obtained by the analysis unit 4. In the present technology, the configuration of the output unit 5 is not particularly limited. For example, the output unit 5 may be configured to generate, only in the case where an abnormal analysis result is obtained during measurement, a notification signal at a specific time, and notify a user of the result in real time. With this configuration, since the user is notified of the analysis result only at the specific time point when the abnormal analysis result has been determined, the usability is improved.

Further, also the method of notifying the user is not particularly limited. For example, the user may receive the notification via the display unit 6 to be described later, a display, a printer, a speaker, lighting, or the like. Further, for example, a device having a communication function of transmitting an e-mail or the like for notifying that a notification signal has been generated to a mobile device such as a cellular phone and a smartphone may be used together with the output unit 5.

Further, in the present technology, the output unit 5 may have a function of notifying, in the case where, for example, the plurality of electrical measurement containers 101 including the above-mentioned two or more types of blood clotting-related assays are not set in the apparatus 100 although analyzing the magnitude of a thrombosis risk is input to the apparatus 100 in advance, the user of a warning or the like to prompt the user to set the container 101.

Further, in the present technology, the output unit 5 may output the magnitude(s) of a thrombosis risk and/or a bleeding risk on the basis of the analysis result of the analysis unit 4. As a result, by using the blood-clotting-system analysis apparatus according to the embodiment of the present technology, it is possible to thoroughly find even a sample (blood sample) having both of the thrombosis risk and the bleeding risk, leading to early treatment.

Further, in the present technology, the output unit 5 may further output the cause(s) of the thrombosis risk and/or the bleeding risk on the basis of the analysis result of the analysis unit 4. As a result, it is possible to not only determine each risk but also know the cause of the risk, and thus, the needs of medical sites where quickness is necessary can be met.

Note that the cause of the thrombosis risk may be due to, for example, any one or more selected from the group consisting of exposure of the tissue factor to the blood sample, reduction in the amount of antithrombin or antithrombin reaction rate, and enhancement of the intrinsic clotting pathway. Further, the cause of the bleeding risk may be due to, for example, deficiency of the extrinsic clotting factor and/or deficiency of the intrinsic clotting factor. In other words, in the present technology, the thrombosis risk and the bleeding risk may have a plurality of causes or only one cause.

(6) Display Unit 6

The display unit 6 displays the analysis result by the analysis unit 4, the data relating to the complex dielectric constant measured by the measurement unit 3, the notification result from the output unit 5, and the like. The configuration of the display unit 6 is not particularly limited. For example, as the display unit 6, a display, a printer, or the like can be adopted. Further, in the present technology, the display unit 6 does not necessarily need to be provided, and an external display apparatus may be connected.

(7) Storage Unit 7

The storage unit 7 stores the analysis result by the analysis unit 4, the data relating to the complex dielectric constant measured by the measurement unit 3, the notification result from the output unit 5, and the like. The configuration of the storage unit 7 is not particularly limited. For example, as the storage unit 7, a hard disk drive, a flash memory, an SSD (Solid State Drive), or the like can be adopted. Further, in the present technology, the storage unit 7 does not necessarily need to be provided, and an external storage apparatus may be connected.

Further, in the present technology, the operation program or the like of the blood-clotting-system analysis apparatus 100 may be stored in the storage unit 7.

(8) Measurement Condition Control Unit 8

The measurement condition control unit 8 controls the measurement time and/or the measurement frequency, and the like in the measurement unit 3. As a specific method of controlling the measurement time, the measurement interval can be controlled depending on the amount of data necessary for the target analysis, and the like, or the timing of finishing the measurement can be controlled in the case where, for example, the measurement value has been substantially leveled off.

Further, it is also possible to control the measurement frequency depending on the type of the blood sample B to be measured, the measurement value necessary for the target analysis, and the like. Examples of the method of controlling the measurement frequency include a method of changing the frequency of alternating voltage to be applied between the electrodes 1a and 1b, and a method of superimposing a plurality of frequencies to measure the impedance at the plurality of frequencies. Examples of the specific method include a method of arranging a plurality of single-frequency analyzers side by side, a method of sweeping a frequency, a method of superimposing frequencies and extracting information of each frequency with a filter, and a method of performing measurement by using the response to impulse.

(9) Temperature Control Unit 9

The temperature control unit 9 controls the temperature in the electrical measurement container 101. In the blood-clotting-system analysis apparatus 100 according to the embodiment of the present technology, this temperature control unit 9 does not necessarily need to be provided. However, in order to keep the blood sample B to be analyzed in an optimal condition for measurement, it is favorable to provide the temperature control unit 9.

Further, in the case of providing the sample standby unit 14 as will be described later, the temperature control unit 9 may control the temperature in the sample standby unit 14. Further, in the case where an agent is put in the blood sample B at the time of or before the measurement, the temperature control unit 9 may be provided to control the temperature of the agent. In this case, the temperature control unit 9 may be provided for the temperature control in the electrical measurement container 101, the temperature control in the sample standby unit 14, and the temperature control of the agent. Alternatively, one temperature control unit 9 may perform all the temperature control.

The specific method of controlling the temperature is not particularly limited. However, for example, by providing the container holding unit 103 with a temperature adjustment function, the container holding unit 103 can be made function as the temperature control unit 9.

(10) Blood Sample Supply Unit 10

The blood sample supply unit 10 automatically supplies the electrical measurement container 101 with the blood sample B. In the blood-clotting-system analysis apparatus 100 according to the embodiment of the present technology, this blood sample supply unit 10 does not necessarily need to be provided. However, by providing the blood sample supply unit 8, it is possible to automatically perform in each step of analyzing the blood clotting system.

The specific method of supplying the blood sample B is not particularly limited. However, for example, it is possible to automatically supply the electrical measurement container 101 with the blood sample B by using a pipetter and a tip attached to the end of the pipetter. In this case, in order to prevent measurement errors or the like from occurring, it is favorable that the tip is disposable. Further, it is also possible to automatically supply from the reservoir of the blood sample B to the electrical measurement container 101 by using a pump or the like. Further, it is also possible to automatically supply the electrical measurement container 101 with the blood sample B by using a permanent nozzle or the like. In this case, in order to prevent measurement errors or the like from occurring, it is favorable to provide the nozzle with a cleaning function.

Further, in the present technology, the blood sample supply unit 10 may include a function (barcode reader, etc.) for identifying and automatically reading the type and the like of the blood sample B that is a sample.

(11) Agent Supply Unit 11

The agent supply unit 11 automatically supplies the electrical measurement container 101 with one or more types of agents. In the blood-clotting-system analysis apparatus 100 according to the embodiment of the present technology, this agent supply unit 11 does not necessarily need to be provided. However, by providing the agent supply unit 11, it is possible to automatically perform each step of analyzing the blood clotting system.

The specific method of supplying the agent is not particularly limited, and a method similar to that of the blood sample supply unit 10 described above can be used. In particular, it is favorable to supply the agent by using a method capable of supplying a predetermined amount of agent without being in contact with the electrical measurement container 101. For example, in the case of a liquid agent, the agent can be discharged and supplied. More specifically, for example, it is possible to discharge and supply the liquid agent to the container 101 by introducing the liquid agent into a discharge pipe in advance and blowing, for a short time, pressurized air separately connected via a pipe line connected to the discharge pipe into the pipe line. At this time, by adjusting the air pressure and the valve opening/closing time, it is also possible to adjust the amount of liquid agent to be discharged.

Further, in addition to the blowing of air, it is also possible to discharge and supply the liquid agent to the container 101 by using vaporization of the liquid agent itself or air dissolved in it by heating. At this time, it is also possible to adjust the volume of generated bubbles and adjust the amount of liquid agent to be discharged by adjusting the voltage applied to a vaporizing chamber in which a heating element or the like is placed and the application time.

Further, it is also possible to supply the container 101 with the liquid agent by driving a movable unit provided in the pipe line using a piezoelectric element (piezo element) or the like without using air, and delivering the liquid agent in an amount determined by the volume of the movable unit. Further, for example, it is also possible to supply the agent by using a so-called inkjet method in which a liquid agent is made into fine droplets and sprayed directly onto the desired container 101.

Further, in the present technology, the agent supply unit 11 may be provided with a stirring function, a temperature control function, a function (e.g., barcode reader) for identifying and automatically reading, for example, the type of the agent, and the like.

(12) Precision Management Unit 12

The precision management unit 12 manages precision of the measurement unit 3. In the blood-clotting-system analysis apparatus 100 according to the embodiment of the present technology, this precision management unit 12 does not necessarily need to be provided. However, by providing the precision management unit 12, it is possible to improve the precision of the measurement by the measurement unit 3.

The specific method of managing the precision is not particularly limited, and a well-known precision management method can be freely used as appropriate. Examples of such a method include a method of managing the precision of the measurement unit 3 by performing calibration of the measurement unit 3, such as a method of performing calibration of the measurement unit 3 by placing a metal plate or the like for short-circuiting in the apparatus 100 and short-circuiting the electrode and the metal plate before starting measurement, a method of bringing a calibration jig or the like into contact with the electrode, and a method of performing calibration of the measurement unit 3 by placing a metal plate or the like in a container having the same form as that of the container 101 in which the blood sample B is to be put and short-circuiting the electrode and the metal plate before starting measurement.

Further, the present technology is not limited to the above-mentioned methods, and a free method, e.g., a method of managing the precision of the measurement unit 3 by checking the state of the measurement unit 3 before the actual measurement and calibrating the measurement unit 3 by performing the above-mentioned calibration or the like only when there is an abnormality, may be selected as appropriate and used.

(13) Drive Mechanism 13

The drive mechanism 13 is used for moving the electrical measurement container 101 in the measurement unit 3 depending on various purposes. For example, by moving the container 110 to the direction of changing the direction of gravity applied to the blood sample B held in the container 110, it is possible to prevent the measurement value from being affected by sedimentation of the sedimentation component in the blood sample B.

Further, for example, it is possible to drive the electrical measurement container 101 so that the application unit 2 and the electrodes 1a and 1b can be disconnected from each other at the time of non-measurement and the application unit 2 and the electrodes 1a and 1b can be electrically connected to each other at the time of measurement.

Further, for example, in the case of providing a plurality of electrical measurement containers 101, by configuring the containers 101 to be capable of moving, it is possible to perform measurement, blood sample supply, agent supply, and the like by moving the containers 110 to necessary sections. That is, since it is unnecessary to move the measurement unit 3, the blood sample supply unit 10, the agent supply unit 11, and the like to the target electrical measurement container 101, it is unnecessary to provide a drive unit or the like for moving the respective units and it is possible to miniaturize the apparatus and reduce the cost.

(14) Sample Standby Unit 14

The sample standby unit 14 causes the isolated blood sample B to stand by before measurement. In the blood-clotting-system analysis apparatus 100 according to the embodiment of the present technology, this sample standby unit 14 does not necessarily need to be provided. However, by providing the sample standby unit 14, it is possible to smoothly measure the dielectric constant.

In the present technology, the sample standby unit 14 may be provided with a stirring function, a temperature control function, a mechanism for moving to the electrical measurement container 101, a function (e.g., barcode reader) for identifying and automatically reading, for example, the type of the blood sample B, an automatic opening function, and the like

(15) Stirring Mechanism 15

The stirring mechanism 15 stirs the blood sample B, and stirs the blood sample B and an agent. In the blood-clotting-system analysis apparatus 100 according to the embodiment of the present technology, this stirring mechanism 15 does not necessarily need to be provided. However, for example, in the case where the blood sample B contains a sedimentation component or the case where an agent is added to the blood sample B at the time of measurement, it is favorable to provide the stirring mechanism 15.

The specific stirring method is not particularly limited, and a well-known stirring method can be freely used as appropriate. Examples of such a method include stirring by pipetting, stirring using a stirring rod, a stirring bar, or the like, and stirring by reversing the container containing the blood sample B or the agent.

(16) User Interface 16

The user interface 16 is a section for a user to operate. The user is capable of accessing the respective units of the blood-clotting-system analysis apparatus 100 via the user interface 16.

(17) Server 17

The server 17 includes at least a storage unit that stores the data acquired by the measurement unit 3 and/or the analysis result acquired by the analysis unit 4, and is connected to at least the measurement unit 3 and/or the analysis unit 4 via a network.

Further, the server 17 is capable of managing various types of data uploaded from the respective units of the blood-clotting-system analysis apparatus 100 and outputting the various types of data to the display unit 6 or the like in response to an instruction from the user.

(18) Others

Note that functions performed by the respective units of the blood-clotting-system analysis apparatus 100 according to the embodiment of the present technology may be stored as a program in a personal computer or a hardware resource including a control unit including a CPU and the like and a recording medium (non-volatile memory (USB memory or the like), HDD, CD, and the like), and implemented by the personal computer or the control unit.

2. Blood-Clotting-System Analysis Method

A blood-clotting-system analysis method includes at least an application step, a measurement step, and an analysis step. Further, the blood-clotting-system analysis method may include another step as necessary. Hereinafter, each step will be described in detail.

(1) Application Step

The application step includes applying alternating voltage to a pair of electrodes at predetermined time intervals. The detailed method is the same as the above-mentioned method performed by the application unit 2, and thus, description thereof is omitted here.

(2) Measurement Step

The measurement step includes measuring the complex dielectric constant of a blood sample placed between the pair of electrodes. The detailed method is the same as the above-mentioned method performed by the measurement unit 3, and thus, description thereof is omitted here.

(3) Analysis Step

The analysis step includes analyzing, by using at least the two or more types of blood clotting-related assays, activity of a clotting factor and activity of a clotting inhibition factor on the basis of the complex dielectric constant of a particular frequency in a predetermined time period measured at the above-mentioned time intervals after the anticoagulant effect acting on the blood sample is resolved. The detailed method is the same as the above-mentioned method performed by the analysis unit 4, and thus, description thereof is omitted here.

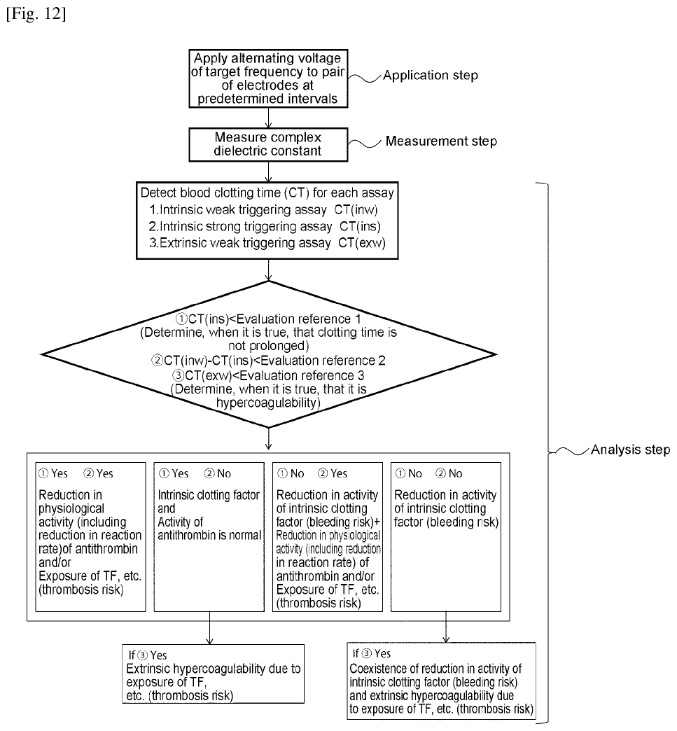

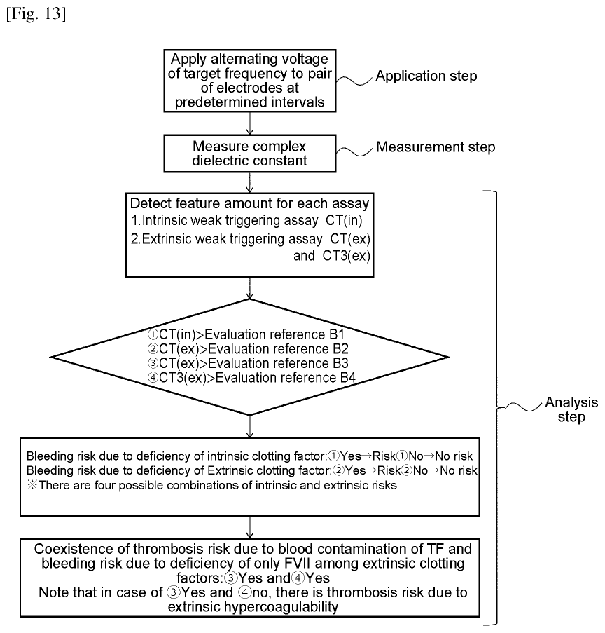

Note that a more specific analysis method is shown in FIG. 12 of Example 3 or FIG. 13 of Example 4 described below.

### EXAMPLES

Hereinafter, the present technology will be described in more detail on the basis of Examples.

Note that Examples described below show an example of a typical embodiment of the present invention, and the scope of the present technology is not narrowly interpreted by Examples.

### Example 1

Blood of 24 adult patients who are going to undergo cardiovascular surgery using cardiopulmonary bypass was measured.

<Sample>

The timing of blood collection is as follows. Blood was collected into a blood collection tube containing citric acid as an anticoagulant.

(A) After induction of anesthesia and before start of surgery

(B) At time when heparin neutralization with protamine is finished after cardiopulmonary bypass

(C) At time when entering ICU after surgery

(D) One week after surgery

(E) One month after surgery

<Measurement>

In addition to DBCM (dielectric blood coagulometry) measurement, thromboelastometry, a screening blood clotting test, a quantitative test of clotting factors, a thrombin generation test, and the like were performed.

For the DBCM measurement, a dielectric coagulometer (prototype machine for experiment) (manufactured by Sony Corporation) was used. The measurement system includes an automatic blood dispensing unit, a quadruple cartridge holder controlled at 37° C. (within +0 or −1° C.), an impedance analyzer board (frequency range: 100 Hz to 40 MHz) connected to a disposable cartridge, and a PC. The cartridge is formed of polypropylene, and a pair of electrodes each formed of titanium are inserted thereinto. The cartridge is capable of measuring the complex dielectric constant of blood by functioning as a parallel plate capacitor. Further, the pair of electrodes are arranged so as to be less susceptible to blood deposition. A cartridge in which a reagent is encapsulated in advance is set to a sample cartridge holder. Note that the dielectric coagulometer (prototype machine for experiment) used this time includes a quadruple sample cartridge holder, and is capable of simultaneously performing four types of measurement using different reagents.

The reagents used in this Example 1 and the names of the assays corresponding thereto are shown below: an assay EX in which the extrinsic clotting pathway is activated by a tissue factor; an assay IN in which the intrinsic clotting pathway is activated by ellagic acid; an assay PI in which the extrinsic clotting pathway is activated by a tissue factor in the state where platelet aggregation is inhibited by cytochalasin D; and an assay LI in which the extrinsic clotting pathway is activated by a tissue factor in the state where a fibrinolytic system is inhibited by aprotinin. Note that in addition to these reagents, calcium chloride was also commonly added to resolve the anticoagulant effect by citric acid. Further, the sample was used for measurement as whole blood without special treatment.

The thromboelastometry measurement was performed using ROTEM delta (manufactured by TEM innovations GmBH) in accordance with the procedure specified by the manufacturer. The assays to be used for measurement were selected so as to correspond to the DBCM measurement. Specifically, the assays are an EXTEM assay in which the extrinsic clotting pathway is activated by a tissue factor, an INTEM assay in which the intrinsic clotting pathway is activated by ellagic acid, a FIBTEM assay in which the extrinsic clotting pathway is activated in the state where the platelet aggregation is inhibited by cytochalasin D, and an APTEM assay in which the extrinsic clotting pathway is activated by a tissue factor in the state where a fibrinolytic system is inhibited by aprotinin. Note that the sample was used for measurement as whole blood without special treatment.

A screening blood clotting test and a quantitative test of clotting factors were performed by a blood clotting fibrinolysis measurement apparatus (ACLTOP 300 CTS; manufactured by Instrumentation Laboratory). It is necessary to use plasma from which blood cell components have been removed. For that reason, the blood sample was centrifuged at 3000 rpm×10 min (at 20° C.), the supernatant was recovered, and the supernatant was centrifuged at 3000 rpm×10 min (at 20° C.) to obtain a supernatant. The finally obtained supernatant was used. The plasma thus obtained is one from which substantially all platelets have been removed, and can be regarded as platelet poor plasma (PPP). The measurement was performed using the procedure specified by the manufacture and a dedicated reagent. The measurement items are as shown in Table 1 below.

TABLE 1
[Table 1 Measurement items]

| Measurement item name | Test content |
|---|---|
| PT-RP[INR] | Prothrombin time-international normalized ratio |
| APTT-SS[s] | Activated partial thromboplastin time |
| AT[%] | Antithrombin |
| DDHS500[ng/mL FEU] | D-dimer |
| FII-RP[%] | Thrombin |
| FIX-SS[%] | Factor IX |
| FV-RP[%] | Factor V |
| FVII-RP[%] | Factor VII |
| FVIII-SS[%] | Factor VIII |
| FX-RP[%] | Factor X |
| FXI-SS[%] | Factor XI |
| FXII-SS[%] | Factor XII |
| FXIII Ag[%] | Factor XIII |
| Fib-C[mg/dL] | Fibrinogen |
| LIQ HEP[IU/mL] | Residual heparin |
| PI[%] | Plasmin inhibitor |
| PLG[%] | Plasminogen |
| VWF: Ag[%] | Von Willebrand factor |
| FDP[μg/mL] | Fibrin degradation product |

<Results and Discussions>

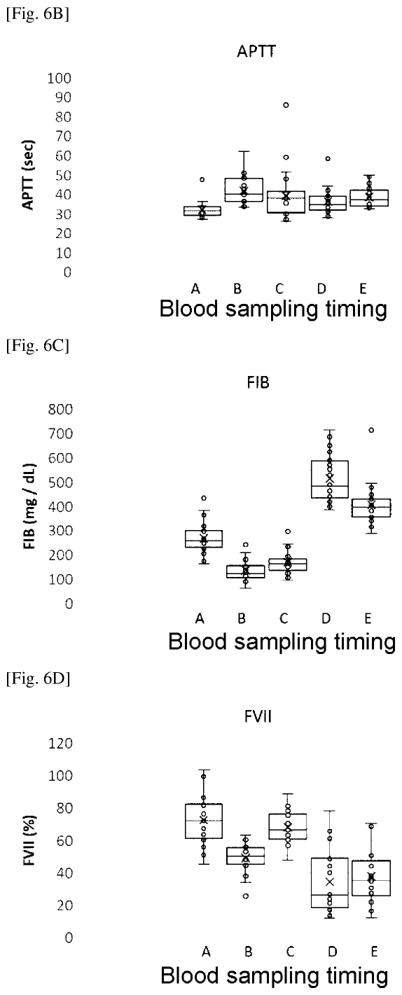

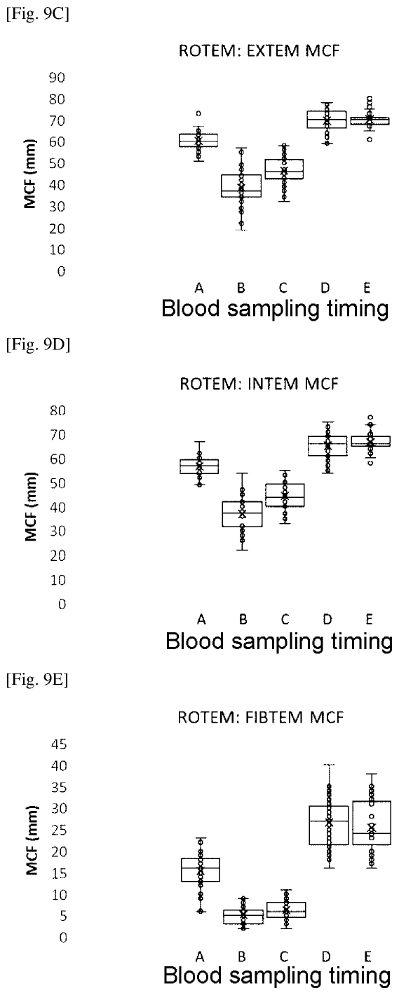

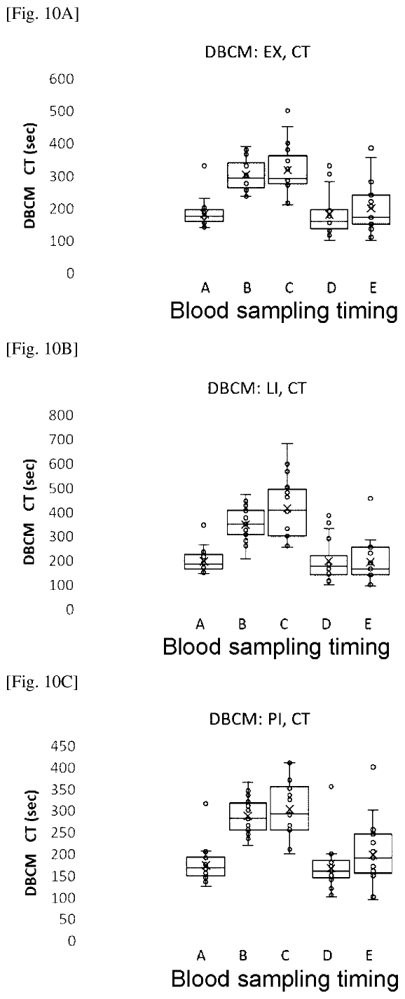

In the extrinsic clotting tests, i.e., PT-INR (FIG. 6A) and ROTEM EXTEM (FIG. 8A), the blood clotting time was extended after cardiopulmonary bypass was finished, as compared with that at the time of induction of anesthesia immediately before surgery, but the extension tendency was eased at the time when entering the ICU after surgery was finished. Of particular note is that the blood clotting times one week after surgery and one month after surgery were extended as compared with the blood clotting time immediately before surgery. One reason for this is that FVII located upstream of the extrinsic clotting pathway one week after surgery and one month after surgery is considerably reduced (FIG. 6D).

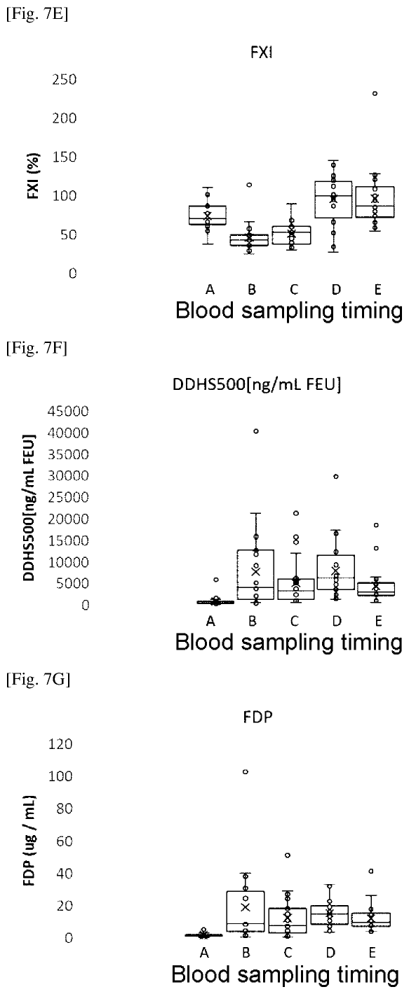

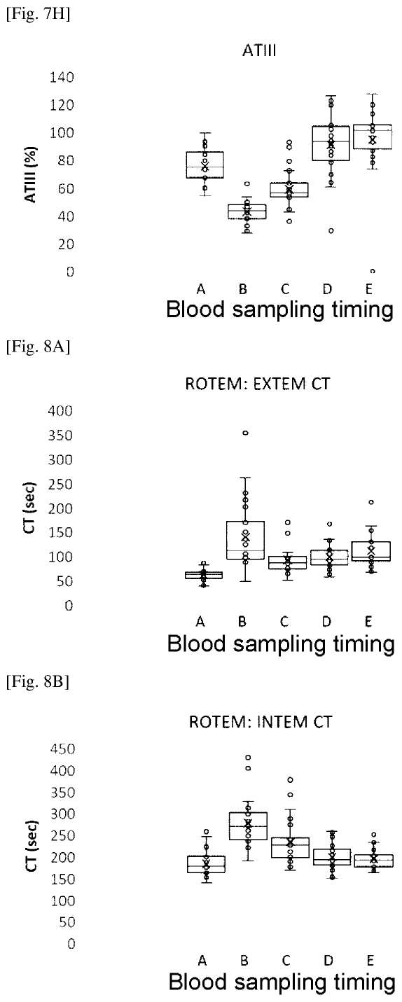

Focusing on the change in each clotting factor, many of these factors have a low value after cardiopulmonary bypass is finished (FIGS. 6C and 6D, and FIG. 7E), and blood dilution by infusion also contributes thereto. For the factors one week after surgery and one month after surgery, the tendency thereof differed depending on the measurement item. For example, fibrinogen had a high value particularly one week after surgery due to post-operative inflammation. Meanwhile, it can be said that FVII had a considerably low value as compared with the change tendency of other factors (FIG. 6D). The fibrinolytic indexes, i.e., DD (FIG. 7F) and FDP (FIG. 7G), had a high value after cardiopulmonary bypass was finished, and tended to be higher than those immediately before surgery, even one month after surgery. Further, AT (FIG. 7H) was reduced with surgery, but recovered one week after surgery.

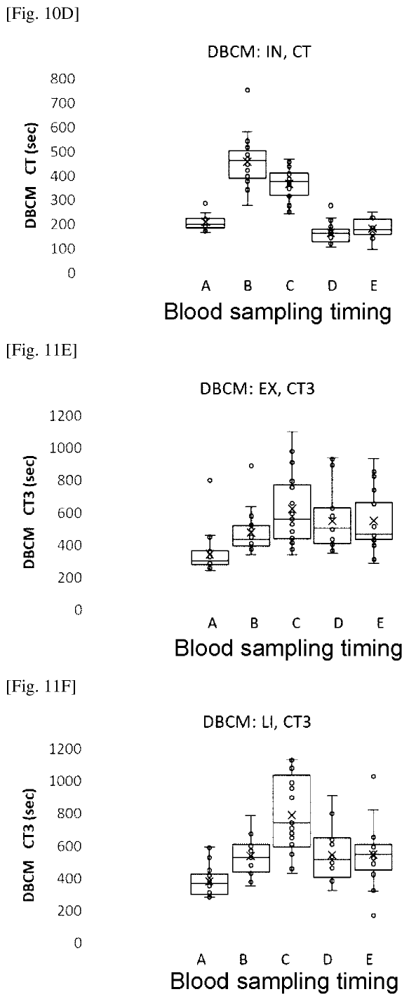

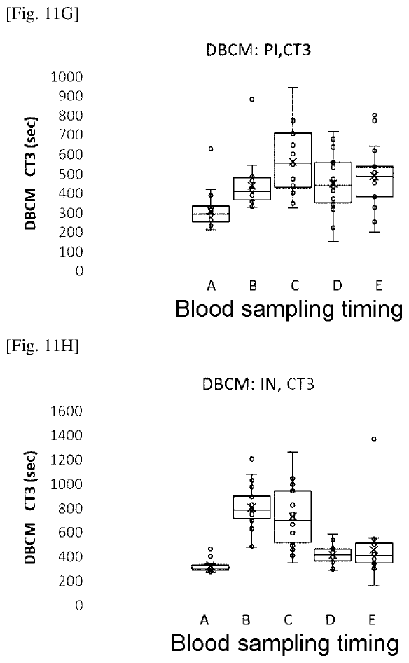

Meanwhile, regarding DBCM, as a feature amount of the dielectric constant change of 10 MHz, the results obtained by performing, for each assay and blood collection timing, analysis and summarization with a time that gives the minimum value as a time CT and a time at which the slope of the increase in the dielectric constant with blood clotting is the largest (time that gives the largest slope) as a time CT3 are shown in FIG. 10A to FIG. 11H. At the time when entering the ICU after cardiopulmonary bypass is finished, the extension tendency was generally shown as compared with that immediately before surgery. Such a change matches the result of PT-INR or ROTEM EXTEM shown above.

However, after that (one week after surgery and one month after surgery), the level of the time CT of each of the assays (EX, LI, and PI) that trigger the extrinsic clotting pathway is has returned to that immediately before surgery, and the time CT of some samples has been reduced. From this, it was found that a thrombosis risk (hypercoagulability) that could not be found by PT-INR or ROTEM EXTEM could be evaluated.

Meanwhile, in the assays (EX, LI, and PI) that trigger the extrinsic clotting pathway, the extension tendency has been found in the time CT3 one week after surgery or one month after surgery rather than immediately before surgery, which matches the result of PT-INR or ROTEM EXTEM. In other words, it can be said that a bleeding risk due to reduction in the amount of FVII is reflected.

Specifically, it was found that both the thrombosis risk and the bleeding risk exist one week after surgery or one month after surgery, which could not be recognized by the existing normal test. In a normal test (e.g., PT-INR or EXTEM), the clotting time is prolonged, and thus, a thrombosis risk is overlooked. In fact, it is believed that a potential thrombosis risk due to exposure of blood to a trace tissue factor is increased.

Note that since the amount of FVII is reduced, it is believed that the speed at which the clotting reaction accelerates is slow. However, there still remains sufficient FVII for the clotting reaction to proceed. As compared with the fact that there is little risk of the clotting reaction to proceed in healthy blood vessels because there is little exposure of the tissue factor in healthy persons, it can be said that the measurement results one week after surgery and one month after surgery indicate a high thrombosis risk. Meanwhile, once blood vessels are cleaved and bleeding occurs, the reduction in the amount of FVII takes time for hemostasis. Even if hemostasis is eventually reached, it may be fatal because a rapid clotting reaction does not occur. During bleeding, blood is exposed to the excess amount of tissue factor expressed extravascularly, and thus, a clotting reaction rapidly proceeds in a healthy person (with no reduction in the amount of FVII). However, in the samples corresponding to the measurement results one week after surgery and one month after surgery, it takes time for hemostasis even blood is exposed to the excess amount of tissue factor due to reduction in the amount of FVII.

Further, if it is possible to confirm that there is no abnormality in the intrinsic pathway by performing intrinsic assay IN, it is supported that both of the above-mentioned thrombosis risk and bleeding risk are due to the extrinsic pathway, i.e., exposure of the trace tissue factor to blood and reduction in the amount of FVII by the exposure. Further, it is possible to distinguish them also from the thrombosis risk due to the reduction in activity of antithrombin described in Example 2 described below.

Focusing on the measurement result of each clotting factor again on the basis of the abovementioned findings, it can be inferred that the result does not contradict the evaluation by DBCM that a thrombosis risk is increased due to exposure of blood to the trace tissue factor. Specifically, although the amount of FVII is considerably reduced one week after surgery and one month after surgery, there is almost no such a tendency for other factors. Such a result is worthy of note in the following point. Specifically, the possibility that the reduction in the amount of FVII is consumptive cannot be ignored because it is unlikely that only the hepatic synthesis of FVII is selectively reduced as compared with other clotting factors. It is believed that a very low concentration of tissue factors is continuously exposed to blood to the extent that thromboembolic events do not occur. At such a low concentration, the tissue factor does not immediately appear as thrombosis or disseminated intravascular coagulation (DIC). However, it is believed that the tissue factor binds to FVII or FVIIa, which causes consumptive reduction in the amount of FVII.

One of the reasons why such a risk cannot be evaluated by a PT-INR assay or ROTEM EXTEM assay is considered to be due to a high concentration of tissue factor used for the test. The final concentration of the tissue factor in the EXTEM assay is estimated to be approximately 30 times the concentration in DBCM performed this time, and the final concentration in the PT-INR assay is estimated to be approximately 1,000 times as the concentration in DBCM performed this time. An excess amount of tissue factor as compared with the amount of tissue factor in blood is input as a test reagent in these assays. For that reason, it is believed that the hypercoagulability effect by the tissue factor in blood is buried, and conversely, the influence of the tendency of FVII factor deficiency on the test becomes dominant, which is observed as clotting extension. Further, in ROTEM, as a characteristic of viscoelasticity measurement, soft fibrin gel seen in the initial stage of a clotting reaction is broken, and such an initial clotting reaction cannot be detected. Therefore, it is believed that in the case where the sample that originally has a short clotting start time and is in the hypercoagulability state does not rapidly transit to hard gel due to the low level of FVII, the hypercoagulability of the sample cannot be evaluated.

From the above, it was suggested that the following requirements should be recommended in order to evaluate a thrombosis risk with high precision.

In the apparatus, a method that does not break initial fibrin gel without applying shear stress to blood being measured, such as a DBCM, is favorable. On the contrary, a method that breaks initial fibrin gel during measurement, such as thromboelastometry, is not favorable. Further, as the reagent, a tissue factor at a concentration of very weakly triggering the extrinsic clotting pathway is used. The analysis unit of the apparatus evaluates a thrombosis risk (favorably, a thrombosis risk and a bleeding risk) on the basis of the complex dielectric constant of a particular frequency in a predetermined time period measured at the above-mentioned time intervals after the anticoagulant effect acting on the blood sample is resolved. In this analysis, it is favorable to use the feature amount extracted from the complex dielectric constant spectrum of the particular frequency.

Note that the above-mentioned estimation is finally obtained when considering the present technology by carrying out tests of a large number of items, and it is very difficult to perform similar estimation without the present technology. Further, in the normal medical practice, it is difficult to perform such exhaustive tests and estimation based on the tests in view of the medical economic aspect.

### Example 2

Regarding physiological activity of antithrombin, which is difficult to evaluate in the existing test, research was conducted on an evaluation method for a thrombosis risk, a bleeding risk, and coexistence of both risks. The change in association with preservation of blood of a healthy person was used as a verification model in Experiment 1, and it was verified in Experiment 2 that addition of antithrombin to blood of the healthy person extended the clotting time.

<Experimental Method>

The Experimental Method of Experiment 1 is described below. Venous blood of a healthy person was collected by vacuum blood collection tubes using citric acid as an anticoagulant. The first one was discarded, and the blood collected in the second tube and the third tube was used for experiment. Of the two blood collection tubes to be used for experiment, the first one was used for experiment within one hour after blood collection. The other tube was preserved unopened at room temperature (25° C.) for 25 hours, and then used for experiment. The measurement was performed for DBCM, PT-INR, APTT, and antithrombin activity. Among them, the measurement other than DBCM measurement was performed using plasm obtained by centrifuging the blood. Further, the assay used in the DBCM measurement includes a CA assay, an IN2 assay, an IN4 assay, and an IN20 assay. Note that the CA assay is one in which only a calcium salt is used as a reagent. Further, in the IN2 assay, the IN4 assay, and the IN20 assay, a calcium salt and ellagic acid that is a clotting initiator of the intrinsic clotting pathway are used as reagents. Specifically, as the ellagic acid, a six-fold dilution of actinFSL (reagent for APTT measurement) purchased from Sysmex Corporation with PBS was used in an amount of 1.2, 2.4, and 12 μL for 200 μL of blood.

The Experimental Method of Experiment 2 is described below. Blood of a healthy person collected similarly to Experiment 1 to which antithrombin purchased as a reagent was added, and blood of a healthy person collected similarly to Experiment 1 to which physiological saline had been added as a control were prepared to perform experiment. The measurement was performed for DBCM, APTT, and antithrombin activity. Among them, in the DBCM measurement, the CA assay and the IN20 assay were performed.

<Results and Discussions>

The Results and Discussion of Experiment 1 is described below. The response obtained by the DBCM measurement was analyzed focusing on, for example, the time CT0 (time that gives the maximum value of the complex dielectric constant at 1 MHz) and the time CT (time that gives the minimum value of the complex dielectric constant at 10 MHz), and the results were shown in Table 2 below. The measurement results of PT-INR, APTT, and antithrombin activity were also summarized in Table 2 below.

TABLE 2
[Table 2 Blood preservation at room temperature
and clotting-related test result]

| | | | After presentation |
|---|---|---|---|
| Sample | | Before preservation | for 25 hours |
| CA | CT (sec) | 375 | 240 |
| CA | CT0 (sec) | 1830 | 710 |
| IN2 | CT (sec) | 350 | 235 |
| IN2 | CT0 (sec) | 770 | 545 |
| IN4 | CT (sec) | 300 | 190 |
| IN4 | CT0 (sec) | 535 | 190 |
| IN20 | CT (sec) | 145 | 150 |
| IN20 | CT0 (sec) | 165 | 140 |
| PT-INR | | 1.04 | 1.08 |
| APTT | (sec) | 26.7 | 29.7 |

| | | |
|---|---|---|
| AT III activity | 107% | 111% |

There is no change in antithrombin activity after approximately 24 hours of blood preservation in accordance with the literature (Ann Clin Biochem. 2017 July; 54(4):448-462), but it has been reported that the amount of FVIII is reduced due to inhibition by protein C (literature (Feng et al., Scientific Reports, 2014, 4: 3868)). Other clotting factors include those in which the amount of FVIII is slightly reduced and those in which the amount of FVIII is not significantly reduced. In other words, it has been generally recognized that the clotting ability is slightly reduced as a whole because antithrombin activity, which is a clotting inhibition system, does not change and some factors of the clotting system are reduced.

Among the experimental results shown in Table 2 above, the antithrombin activity is not reduced even after the blood preservation, and the time of PT-INR and APTT is extended by the blood preservation, which matches the existing general recognition described above. However, considering the measurement results of DBCM, the time CT and the time CT0 are significantly reduced by the blood preservation in the CA assay in which no artificial acceleration of a clotting reaction is performed, and the IN2 assay and IN4 assay in which the intrinsic clotting pathway is very weakly triggered. This means that the reduction in reaction rate of antithrombin is dominant over the reduction in clotting reaction with the blood preservation. In terms of whether or not thrombosis occurs in blood vessels in vivo, it is a problem whether or not very weak triggering of a clotting reaction leads to thrombus formation. In this case, it can be said that the reduction in reaction rate of antithrombin is very important factor. However, such findings have not been obtained until now.

Meanwhile, it was also found that the reduction in reaction rate of antithrombin was not detected if artificial clotting activation was enhanced to the extent of that in the IN20 assay. Specifically, when triggering of the clotting reaction is strong, the activity of the clotting factor becomes dominant.

Further, in order to quantitatively evaluate the reduction in reaction rate of antithrombin, it only needs to perform measurement under a plurality of conditions for triggering blood clotting and check how much the difference in the clotting time depending on the magnitude of the triggering. For example, it is recommended to perform the following analysis. In the measurement, both of the IN20 assay or an assay that triggers a blood clotting reaction more strongly than the IN20 assay and the CA assay or an assay that does not strongly trigger a blood clotting reaction, such as the IN2 assay and the IN4 assay, are performed. Then, the overall activity of the blood clotting factor is evaluated from the clotting time (e.g., CT) of the strong triggering assay. If the time is longer than the reference, it can be seen that the blood clotting ability is reduced. Next, the clotting time of the weak triggering assay is evaluated. If the time is sufficiently extended than the strong triggering assay, it can be seen that there is no need to worry about the reduction in activity of antithrombin. Conversely, when the degree of extension is low, the following three possibilities (and combinations thereof) can be considered as factors of a thrombosis risk. Specifically, the factors of a thrombosis risk correspond to one or more of the reduction in activity of antithrombin, intrinsic hypercoagulability, and the case where the reaction of the extrinsic pathway proceeds as compared with a reaction by the intrinsic weak triggering assay because the amount of exposure of the tissue factor to blood shown in the above-mentioned Example 1 is large.

FIG. 12 is a block diagram showing an example of a blood clotting analysis method in consideration of thoroughly finding a thrombosis risk. Note that in FIG. 12, an evaluation reference 1, an evaluation reference 2, and an evaluation reference 3 can be determined in advance on the basis of the measurement results of a certain number of healthy persons and patients. Further, these references may be set simply as threshold values. However, more favorably, the references may each be given as a function of the blood clotting time (s) measured using a strong triggering assay and the blood clotting time (w) using a weak triggering assay.

As shown in FIG. 12, after the application step and the measurement step, the time

CT is detected for each assay. Specifically, for example, an intrinsic weak triggering assay CT (inw), an intrinsic strong triggering assay CT (ins), and an extrinsic weak triggering assay CT (exw) are detected. After that, determination is performed using the evaluation references set in advance. Specifically, for example, (i) whether or not the assay CT (ins) is smaller than the evaluation reference 1, (ii) whether or not the value obtained by subtracting the assay CT (ins) from the assay CT (inw) is smaller than the evaluation reference 2, and (iii) whether or not the assay CT (exw) is smaller than the evaluation reference 3 are determined.

Then, using these determination results, it is determined that there is reduction in antithrombin physiological activity (including reduction in reaction rate) and/or exposure or the like of a tissue factor (thrombosis risk) in the case where the above-mentioned (i) and (ii) are Yes, and it is determined that there is no abnormality in the intrinsic pathway and antithrombin in the case where the above-mentioned (i) is Yes and the above-mentioned (ii) is No. In this case, if the above-mentioned (iii) is Yes, it is determined that there is extrinsic hypercoagulability (thrombosis risk) due to exposure or the like of a tissue factor.

Further, it is determined that there is reduction in activity of an intrinsic clotting factor (bleeding risk)+reduction in antithrombin physiological activity (including reduction in reaction rate) and/or exposure or the like of a tissue factor (thrombosis risk) in the case where the above-mentioned (i) is No and the above-mentioned (ii) is Yes, and it is determined that there is reduction in activity of an intrinsic clotting factor (bleeding risk) in the case where the above-mentioned (i) and (ii) are No. in this case, if the above-mentioned (iii) is Yes, it is determined that reduction in activity of an intrinsic clotting factor (bleeding risk) and extrinsic hypercoagulability (thrombosis risk) due to exposure or the like of a tissue factor coexist.

As described above, it was shown that reduction in reaction rate of antithrombin due to blood preservation could be evaluated by the CA assay of DBCM or an assay that very weakly triggers a clotting reaction. It is also assumed that the reduction in reaction rate of antithrombin occurs due to some causes in vivo. It is difficult to evaluate the increase in thrombosis risk due to such causes by the existing tests or combinations thereof.

The Results and Discussion of Experiment 2 is described below. The measurement results are summarized in Table 3 below.

TABLE 3
[Table 3 Antithrombin addition test]

| | CA | IN20 | APTT | AT III |
|---|---|---|---|---|
| Sample | CT1(sec) | CT1(sec) | (sec) | activity |
| Whole blood of healthy | 380 | 195 | 26.5 | 85% |
| person + saline(15%) | | | | |
| Whole blood of healthy | 480 | 240 | 51.3 | Not less |
| person + | | | | than 150% |
| antithrombin(15%) | | | | (range over) |

As shown in Table 3 above, it was confirmed that the blood clotting time was extended by adding antithrombin in excess.

### Example 3

As described above, in Example 1, it was shown that even in the case where the sample had both a thrombosis risk and a bleeding risk due to exposure of a tissue factor and reduction in the amount of FVII by the exposure, both of the risks could be evaluated from, for example, evaluation of the extrinsic assays CT and CT3 by the DBCM measurement. Further, in Example 2, a method of evaluating a thrombosis risk due to reduction in activity or the like of antithrombin was shown, and it was shown that the determination thereof could be performed as shown in FIG. 12. However, in FIG. 12, it is difficult to completely determine whether the factors of the thrombosis risk and/or the bleeding risk are in the extrinsic pathway, the intrinsic pathway, or AT.

In this regard, assuming a plurality of cases including the case where the cause of a thrombosis risk is due to exposure of a tissue factor (TF+) and the case where the cause of a thrombosis risk is due to reduction in activity of antithrombin (AT−), how the test results of DBCM change is verified and summarized in Table 4 below. Note that regarding (TF+), also the item “TF+(without reduction in FVII)” is set assuming the case where the reduction in the amount of FVII is not so remarkable.

TABLE 4
[Table 4 Relationship between cause of thrombosis/bleeding risk and DBCM test value]

| | | | | | | |
|---|---|---|---|---|---|---|
| Sample | TF+ | | | | | |
| condition | With reduction in FVII | TF+ | Deficiency of | | Intrinsic | Deficiency if |
| Thrombosis/ | Thrombosis risk + | Without reduction in FVII | extrinsic factor | AT− | hypercoagulability | intrinsic factor |
| bleeding risk | bleeding risk | Thrombosis risk | Bleeding risk | Thrombosis risk | Thrombosis risk | Bleeding risk |

Extrinsic weak triggering assay

| | | | | | | |
|---|---|---|---|---|---|---|
| CT | Reduction | Reduction | Extension | Reduction | Normal | Normal |
| CT3 | Extension | Reduction | Extension | Reduction | Normal | Normal |

Extrinsic strong triggering assay

| | | | | | | |
|---|---|---|---|---|---|---|
| CT | Extension※ | Normal | Extension | Normal | Normal | Normal |
| CT3 | Extension | Normal | Extension | Normal | Normal | Normal |

Intrinsic weak triggering assay

| | | | | | | |
|---|---|---|---|---|---|---|
| CT | Normal | Normal | Normal | Reduction | Reduction | Extension |
| CT3 | Normal | Normal | Normal | Reduction | Reduction | Extension |
| CT0 | Normal | Normal | Normal | Reduction | Reduction | Extension |

Intrinsic strong triggering assay

| | | | | | | |
|---|---|---|---|---|---|---|
| CT | Normal | Normal | Normal | Normal | Reduction※ | Extension |
| CT3 | Normal | Normal | Normal | Normal | Reduction※ | Extension |
| CT0 | Normal | Normal | Normal | Normal | Reduction※ | Extension |

Assay that does not trigger blood clotting reaction (no artificial triggering for re-addition of calcium)

| | | | | | | |
|---|---|---|---|---|---|---|
| CT | Reduction | Reduction | No sensitivity | Reduction | Reduction | No sensitivity |
| CT3 | No sensitivity | Reduction | Extension※ | Reduction | Reduction | Extension※ |

※Low sensitivity

As shown in Table 4 above, by using a plurality of assays, it is possible to perform more detailed analysis regarding a thrombosis risk and/or a bleeding risk, and the cause thereof.

### Example 4

From the results of Example 3, a measurement (evaluation) panel assuming a more specific case was set. More specifically, the case where there is a concern about a bleeding risk (in a perioperative period, during anticoagulation treatment, when there are findings suggesting bleeding, etc.) was assumed.

In this case, it is necessary to perform both an extrinsic assay and an intrinsic assay. Either a weak triggering assay or a strong triggering assay may be used. However, in the case where it is desired to determine whether it is a bleeding risk due to reduction in the amount of FVII factor (due to contamination of a tissue factor) or a bleeding risk due to reduction in the amount of general extrinsic factors, it is better to use a weak triggering assay as the extrinsic assay. In the case where it is not necessary to perform determination to such degree and it is desired to know the test results, a strong triggering assay can be used. As an example, FIG. 13 is a block diagram showing a blood clotting analysis method of determining a bleeding risk using an extrinsic weak triggering assay and an intrinsic weak triggering assay.

As shown in FIG. 13, after the application step and the measurement step, the feature amount is detected for each assay. Specifically, for example, an intrinsic weak triggering assay CT (in) and extrinsic weak triggering assays CT (ex) and CT3 (ex) are detected. Note that the assay CT (in) may be the assay CT3 (in), the assay CT1 (in), or a parameter according to this. Also the assay CT (ex) and the assay CT3 (ex) may each be a parameter according to this.

After that, determination is performed using evaluation references set in advance. Specifically, for example, (i) whether or not the assay CT (in) is larger than an evaluation reference B1, (ii) whether or not the assay CT (ex) is larger than an evaluation reference B2, (iii) whether or not the assay CT (ex) is smaller than an evaluation reference B3, and (iv) the assay CT3 (ex) is larger than an evaluation reference B4 are determined.

Then, using these determination results, it is determined that there is a bleeding risk due to deficiency of an intrinsic clotting factor in the case where the above-mentioned (i) is Yes, it is determined that there is no bleeding risk due to deficiency of an intrinsic clotting factor in the case where the above-mentioned (i) is No, it is determined that there is a bleeding risk due to deficiency of an extrinsic clotting factor in the case where the above-mentioned (ii) is Yes, and it is determined that there is no bleeding risk due to deficiency of an extrinsic clotting factor in the case where the above-mentioned (ii) is No. Four conclusions can be obtained by combining the two determination results. For example, in the case where the above-mentioned (i) and (ii) are Yes, it is determined that there is a bleeding risk due to deficiency of an intrinsic clotting factor and an extrinsic clotting factor.

Further, in the case where the above-mentioned (iii) and (iv) are Yes, it is determined that a thrombosis risk due to contamination of a tissue factor in blood and a bleeding risk due to deficiency of only FVII among extrinsic clotting factors coexist. Note that in the case where the above-mentioned (iii) is Yes and the above-mentioned (iv) is No, it is determined that there is a thrombosis risk due to extrinsic hypercoagulability.

Note that the present technology may also take the configurations.

(1) A blood coagulation system analysis device, comprising:
circuitry configured to:
perform a first blood clotting-related assay on a first blood sample;
perform a second blood clotting-related assay on a second blood sample; and
evaluate an activity of a clotting factor and an activity of a clotting inhibition factor based, at least in part, on an output of the first blood clotting-related assay and an output of the second blood clotting-related assay,
wherein the first blood sample and the second blood sample are distributed from a same blood sample collected from a patient.
(2) The blood coagulation system analysis device of (1), wherein the circuitry is further configured to output information indicating a thrombosis risk and/or information indicating a bleeding risk for the patient based, at least in part on the output of the first blood clotting-related assay and the output of the second blood clotting-related assay.
(3) The blood coagulation system analysis device of (1), wherein the first blood clotting-related assay comprises an extrinsic clotting pathway triggering assay, and the second blood clotting-related assay comprises an intrinsic clotting pathway triggering assay.
(4) The blood coagulation system analysis device of (3), wherein the extrinsic clotting pathway triggering assay comprises an extrinsic weak triggering assay and/or the intrinsic clotting pathway triggering assay comprises an intrinsic weak triggering assay.
(5) The blood coagulation system analysis device of (1) wherein the first and second blood clotting-related assays are selected from the group consisting of an assay that triggers no blood clotting reaction, an intrinsic strong triggering assay, an intrinsic weak triggering assay, an extrinsic strong triggering assay, and an extrinsic weak triggering assay.
(6) The blood coagulation system analysis device of (1), wherein the first blood clotting-related assay comprises a strong triggering assay and the second blood clotting-related assay comprises a weak triggering assay.
(7) The blood coagulation system analysis device of (1), wherein the output of the first blood clotting-related assay and/or the output of the second blood clotting-related assay comprises a blood clotting time.
(8) The blood coagulation system analysis device of (1), wherein
the first blood clotting-related assay comprises an intrinsic weak triggering assay configured to output a first clotting time,
the second blood clotting-related assay comprises an intrinsic strong triggering assay configured to output a second clotting time, and
the circuitry is further configured to perform a third blood clotting-related assay comprising an extrinsic weak triggering assay configured to output a third clotting time.
(9) The blood coagulation system analysis device of (8), wherein determining an activity of a clotting factor and an activity of a clotting inhibition factor comprises:
(i) comparing the first clotting time to a first threshold;
(ii) comparing the second clotting time to a second threshold;
(iii) comparing the third clotting time to a third threshold; and
outputting information indicating a thrombosis risk and/or information indicating a bleeding risk for the patient based, at least in part, on one or more of the comparisons (i), (ii), and (iii).
(10) The blood coagulation system analysis device of (1), wherein the circuitry is further configured to:
determine a first dielectric constant spectrum based on the output of the first blood clotting-related assay;
extract a value for one or more features from the first dielectric constant spectrum;
determine a second dielectric constant spectrum based on the output of the second blood clotting-related assay;
extract a value for the one or more features from the second dielectric constant spectrum; and
evaluate the activity of a clotting factor and the activity of a clotting inhibition factor based on the value extracted for the one or more features from the first dielectric constant spectrum and the value extracted for the one or more features from the second dielectric constant spectrum.
(11) The blood coagulation system analysis device of (10), wherein the one or more features comprises a time CT at which the dielectric constant has a minimum value and a time CT3 at which a slope of the dielectric constant is maximum.
(12) The blood coagulation system analysis device of (1), wherein the circuitry is further configured to:
provide as input to a trained model, information associated with the output of the first blood clotting-related assay and information associated with the output of the second blood clotting-related assay, and
wherein the activity of the clotting factor in the blood sample and the activity of the clotting inhibition factor in the blood sample is based, at least in part, on an output of the trained model.
(13) The blood coagulation system analysis device of (12), wherein
the information associated with the output of the first blood clotting related assay comprises a value for one or more features extracted from a dielectric constant spectrum determined based on the output of the first blood clotting-related assay, and
the information associated with the output of the second blood clotting-related assay comprises a value for the one or more features extracted from a dielectric constant spectrum determined based on the output of the second blood clotting-related assay.
(14) The blood coagulation system analysis device of (12), wherein the trained model comprises a model trained using one or more machine learning techniques.
(15) The blood coagulation system analysis device of (1), wherein the circuitry further comprises:
a measurement unit configured to perform the first and second blood clotting-related assays, the measurement unit configured to measure the dielectric impedance of the first and second blood samples, when each is arranged between electrodes to which an alternating voltage is applied.
(16) A method of analyzing a blood sample to determine an amount of a thrombosis risk and/or an amount of a bleeding risk of a patient associated with the blood sample, the method comprising:
performing a first blood clotting-related assay on a first blood sample;
performing a second blood clotting-related assay on a second blood sample; and
evaluating an activity of a clotting factor and an activity of a clotting inhibition factor based, at least in part, on an output of the first blood clotting-related assay and an output of the second blood clotting-related assay,
wherein the first blood sample and the second blood sample are distributed from a same blood sample collected from a patient.
(17) A blood coagulation analysis system, comprising:
a first electrode and a second electrode arranged opposite the first electrode such that a container including a first blood sample may be arranged between the first and second electrodes;
a voltage generator configured to apply an alternating voltage to the first electrode and the second electrode; and
circuitry configured to:
perform a first blood clotting-related assay on the first blood sample when the alternating voltage is applied to the first and second electrodes;
perform a second blood clotting-related assay on a second blood sample; and
evaluate an activity of a clotting factor and an activity of a clotting inhibition factor based, at least in part, on an output of the first blood clotting-related assay and an output of the second blood clotting-related assay,
wherein the first blood sample and the second blood sample are distributed from a same blood sample collected from a patient.

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

100 Blood-clotting-system analysis apparatus
1a, 1b Pair of electrodes
101 Electrical measurement container
102 Connection unit
103 Container holding unit
2 Application unit
3 Measurement unit
4 Analysis unit
5 Output unit
6 Display unit
7 Storage unit
8 Measurement condition control unit
9 Temperature control unit
10 Blood sample supply unit
11 Agent supply unit
12 Precision management unit
13 Drive mechanism
14 Sample standby unit
15 Stirring mechanism
16 User interface
17 Server

1. A blood coagulation system analysis device, comprising:
circuitry configured to:
perform a first blood clotting-related assay on a first blood sample;
perform a second blood clotting-related assay on a second blood sample; and
evaluate an activity of a clotting factor and an activity of a clotting inhibition factor based, at least in part, on an output of the first blood clotting-related assay and an output of the second blood clotting-related assay,
wherein the first blood sample and the second blood sample are distributed from a same blood sample collected from a patient.

2. The blood coagulation system analysis device of claim 1, wherein the circuitry is further configured to output information indicating a thrombosis risk and/or information indicating a bleeding risk for the patient based, at least in part on the output of the first blood clotting-related assay and the output of the second blood clotting-related assay.

3. The blood coagulation system analysis device of claim 1, wherein the first blood clotting-related assay comprises an extrinsic clotting pathway triggering assay, and the second blood clotting-related assay comprises an intrinsic clotting pathway triggering assay.

4. The blood coagulation system analysis device of claim 3, wherein the extrinsic clotting pathway triggering assay comprises an extrinsic weak triggering assay and/or the intrinsic clotting pathway triggering assay comprises an intrinsic weak triggering assay.

5. The blood coagulation system analysis device of claim 1, wherein the first and second blood clotting-related assays are selected from the group consisting of an assay that triggers no blood clotting reaction, an intrinsic strong triggering assay, an intrinsic weak triggering assay, an extrinsic strong triggering assay, and an extrinsic weak triggering assay.

6. The blood coagulation system analysis device of claim 1, wherein the first blood clotting-related assay comprises a strong triggering assay and the second blood clotting-related assay comprises a weak triggering assay.

7. The blood coagulation system analysis device of claim 1, wherein the output of the first blood clotting-related assay and/or the output of the second blood clotting-related assay comprises a blood clotting time.

8. The blood coagulation system analysis device of claim 1, wherein
the first blood clotting-related assay comprises an intrinsic weak triggering assay configured to output a first clotting time,
the second blood clotting-related assay comprises an intrinsic strong triggering assay configured to output a second clotting time, and
the circuitry is further configured to perform a third blood clotting-related assay comprising an extrinsic weak triggering assay configured to output a third clotting time.

9. The blood coagulation system analysis device of claim 8, wherein determining an activity of a clotting factor and an activity of a clotting inhibition factor comprises:
(i) comparing the first clotting time to a first threshold;
(ii) comparing the second clotting time to a second threshold;
(iii) comparing the third clotting time to a third threshold; and
outputting information indicating a thrombosis risk and/or information indicating a bleeding risk for the patient based, at least in part, on one or more of the comparisons (i), (ii), and (iii).

10. The blood coagulation system analysis device of claim 1, wherein the circuitry is further configured to:
determine a first dielectric constant spectrum based on the output of the first blood clotting-related assay;
extract a value for one or more features from the first dielectric constant spectrum;
determine a second dielectric constant spectrum based on the output of the second blood clotting-related assay;
extract a value for the one or more features from the second dielectric constant spectrum; and
evaluate the activity of a clotting factor and the activity of a clotting inhibition factor based on the value extracted for the one or more features from the first dielectric constant spectrum and the value extracted for the one or more features from the second dielectric constant spectrum.

11. The blood coagulation system analysis device of claim 10, wherein the one or more features comprises a time CT at which the dielectric constant has a minimum value and a time CT3 at which a slope of the dielectric constant is maximum.

12. The blood coagulation system analysis device of claim 1, wherein the circuitry is further configured to:
provide as input to a trained model, information associated with the output of the first blood clotting-related assay and information associated with the output of the second blood clotting-related assay, and
wherein the activity of the clotting factor in the blood sample and the activity of the clotting inhibition factor in the blood sample is based, at least in part, on an output of the trained model.

13. The blood coagulation system analysis device of claim 12, wherein
the information associated with the output of the first blood clotting related assay comprises a value for one or more features extracted from a dielectric constant spectrum determined based on the output of the first blood clotting-related assay, and
the information associated with the output of the second blood clotting-related assay comprises a value for the one or more features extracted from a dielectric constant spectrum determined based on the output of the second blood clotting-related assay.

14. The blood coagulation system analysis device of claim 12, wherein the trained model comprises a model trained using one or more machine learning techniques.

15. The blood coagulation system analysis device of claim 1, wherein the circuitry further comprises:
a measurement unit configured to perform the first and second blood clotting-related assays, the measurement unit configured to measure the dielectric impedance of the first and second blood samples, when each is arranged between electrodes to which an alternating voltage is applied.

16. A method of analyzing a blood sample to determine an amount of a thrombosis risk and/or an amount of a bleeding risk of a patient associated with the blood sample, the method comprising:
performing a first blood clotting-related assay on a first blood sample;
performing a second blood clotting-related assay on a second blood sample; and
evaluating an activity of a clotting factor and an activity of a clotting inhibition factor based, at least in part, on an output of the first blood clotting-related assay and an output of the second blood clotting-related assay,
wherein the first blood sample and the second blood sample are distributed from a same blood sample collected from a patient.

17. A blood coagulation analysis system, comprising:
a first electrode and a second electrode arranged opposite the first electrode such that a container including a first blood sample may be arranged between the first and second electrodes;
a voltage generator configured to apply an alternating voltage to the first electrode and the second electrode; and
circuitry configured to:
perform a first blood clotting-related assay on the first blood sample when the alternating voltage is applied to the first and second electrodes;
perform a second blood clotting-related assay on a second blood sample; and
evaluate an activity of a clotting factor and an activity of a clotting inhibition factor based, at least in part, on an output of the first blood clotting-related assay and an output of the second blood clotting-related assay,
wherein the first blood sample and the second blood sample are distributed from a same blood sample collected from a patient.
